(19) United States
(12) Patent Application Publication
(10) Pub. No.: US 2009/0014010 A1
(43) Pub. Date: Jan. 15, 2009

(54) Tracheostomy Tie System

(76) Inventor: James L. Leckie, Covina, CA (US)

Correspondence Address: KOPPEL, PATRICK & HEYBL, 555 ST. CHARLES DRIVE, SUITE 107, THOUSAND OAKS, CA, 91360, US

(21) Appl. No.: 12/170,255

(22) Filed: Jul. 9, 2008

Related U.S. Application Data
(60) Provisional application No. 60/959,095, filed on Jul. 10, 2007.

(51) Int. Cl. A61M 16/04

(52) U.S. Cl. 128/207.17

(57) ABSTRACT

A tracheostomy tie includes a padded collar with a patient neck engagement side, a strap coupled to the padded collar on a side opposite from the patient neck engagement side and extending from opposite ends of the padded collar. The strap includes a loop material outer surface and tapered distal ends. Tapered connectors are connected to respective tapered distal ends of the strap, each of the tapered connectors having a hook material outer surface. The tapered connectors are operable to pass through respective tracheostomy tube connectors and loop back to engage the loop material so that the padded collar may securely fasten and position a tracheostomy tube to a patient's neck.

Benefit is claimed of Provisional Application Ser. No. 60/959,095 filed Jul. 10, 2007.

### BACKGROUND OF THE INVENTION

1. Field of the Invention

This invention relates to medical devices, and more particularly to ties to secure a tracheostomy tube to a patient's neck.

2. Description of the Related Art

Patient's who have undergone a tracheostomy require a tracheal tube to be securely and comfortably attached to their neck over long periods of time. A tracheal tube assembly is inserted into a hole created in the patient's trachea and often secured to the patient's neck using a tracheostomy tie. Typical tracheostomy ties consist of a variety of materials, such as foam padding, elastic cloth, and wide and narrow materials to maintain a secure fit about the patient's neck while attempting to reduce patient discomfort.

A tracheostomy tie will typically pass around the patient's neck to secure and support the tracheal tube assembly, with each end of the tracheostomy tie fed through eyelets incorporated into either side of the tracheal tube assembly. The ends of the tracheostomy tie often incorporate the use of VELCRO™ material to fold back on itself to secure the tie to the tracheostomy tube assembly.

Unfortunately, long term use of the tracheostomy tie around the patient's neck produces a breakdown of the skin underneath the tracheostomy tie. Moisture produced at the skin/tie interface accelerates breakdown of the skin, in part because of stress-strain deformation of the tie about the patient's neck over time causing localized chafing of the patient's skin. Gauze material is often used between the tracheostomy tube and the patient's skin to reduce this effect by absorbing moisture and increasing the surface area supporting the weight of the tracheostomy tie and tracheostomy tube assembly. Also, because of the need for constant supervision and care for patients using tracheostomy tube assemblies, there is often only one caregiver at a time, making rapid change out of the tracheostomy tie difficult with only two hands to both hold the tracheostomy tube assembly and replace the tie.

A need exists to reduce the breakdown of patient skin underneath the tracheostomy tie during extended wear and to reduce the difficulty associated with replacement of the tracheostomy tie by a single caregiver.

### SUMMARY OF THE INVENTION

A tracheostomy tie, in accordance with one embodiment of the invention, includes a padded collar that has a patient neck engagement side, and a strap coupled to the padded collar on a side opposite from the patient neck engagement side that extends from opposite ends of the padded collar. The strap includes a loop material outer surface and tapered distal ends. Tapered connectors are connected to respective tapered distal ends of the strap and each of the tapered connectors has a hook material outer surface. The tapered connectors are operable to pass through respective tracheostomy tube connectors and loop back to engage the loop material so that the padded collar securely fastens and positions a tracheostomy tube against a patient's neck.

In another embodiment of the invention, a method of assembling a tracheostomy tie includes centering the major axis of a padded collar having a first major axis width with the major axis of a tapered strap that has a second width that is less than the first width, coupling the padded collar to the tapered strap and coupling hook material connectors to respective distal ends of the padded collar.

### BRIEF DESCRIPTION OF THE DRAWINGS

The components in the figures are not necessarily to scale, emphasis instead being placed upon illustrating the principals of the invention. Like reference numerals designate corresponding parts throughout the different views.

FIGS. 1-2 are perspective views of a tracheostomy tie illustrating a back and patient neck engagement sides of the tie, respectively, in accordance with one embodiment of the invention;

FIGS. 3-4 are perspective views of an embodiment of a tracheostomy tie.

FIG. 5 is a cross section view of the tracheostomy tie of FIGS. 3-4 about the line 5-5;

FIG. 6 is an enlarged cross section view of the tracheostomy tie illustrated in FIG. 5 about the line 6-6.

FIG. 7 is a plan view of a tracheostomy tie that has separate strap portions, in accordance another embodiment of the invention.

FIG. 8 is a perspective view of the tracheostomy tie of FIGS. 1-2, illustrating the tie coupled to a tracheostomy tube assembly.

FIG. 9 is a flow diagram describing assembly of one embodiment of a tracheostomy tie.

### DETAILED DESCRIPTION OF THE INVENTION

A tracheostomy tie, in accordance with one embodiment of the invention, includes a padded collar preferably sewn to a strap on a side opposite from the patient neck engagement side, with the strap extending from opposite ends of the padded collar. The strap is preferably made of a loop material outer surface such as double-sided brushed polyester fiber material to work cooperatively with VELCRO™ hook material, and is tapered on its ends. A tapered connector, preferably formed of VELCRO™ hook material, may be sewn to each end of the strap so that the tapered connectors are operable to pass through respective tracheostomy tube assembly connectors (typically eyelets) and back to engage the loop material (See FIG. 2) so that the padded collar securely fastens and positions a tracheostomy tube against the patient's neck.

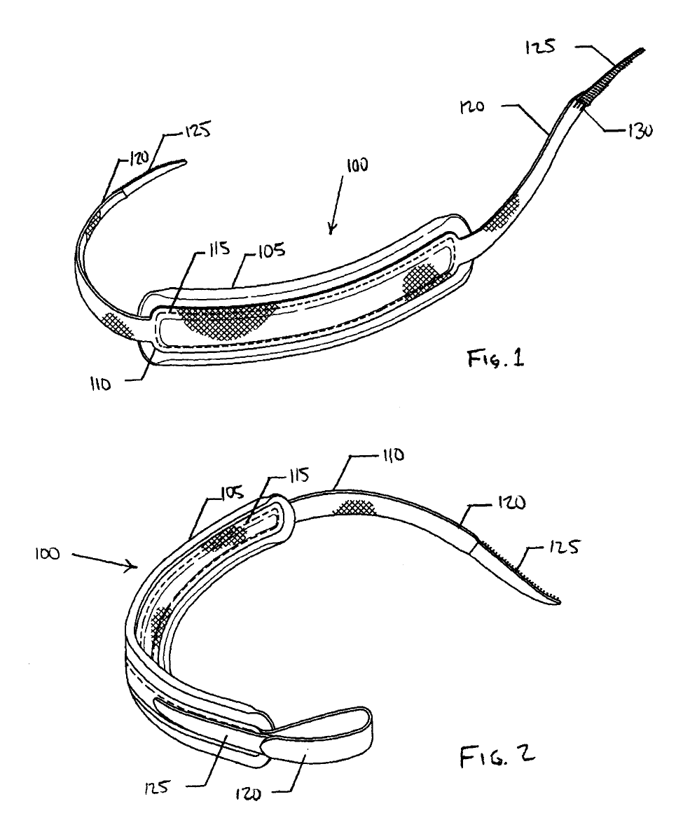

In one embodiment of the invention shown in FIGS. 1-2, a tracheostomy tie 100 has a padded collar 105 coupled to a strap 110, preferably sewn together adjacent longitudinal and lateral sides of the padded collar 105 as indicated with a dashed sew line 115. Although the padded collar 105 and strap 110 are illustrated sized and sewn together at a position set back from a perimeter of the padded collar, the strap 110 may be sewn with other patterns or immediately against the outer perimeter of the padded collar 105. The padded collar 105 and strap 110 may also be coupled using other suitable means, such as with fabric adhesive, by using fusible web techniques, or through the use of fusible material or other flexible glues and tapes.

The strap 110 extends beyond both ends of the padded collar 105 for engagement with tracheostomy tube assembly connectors, with the assembly connectors typically configured as eyelets (not shown). The strap 110 has at least a portion of its outer surface preferably formed of loop material for complementary operation with a hook material connector. The strap 110 is slightly tapered, preferably tapered at each distal end 120 to aid insertion through tracheostomy tube assembly connector, with each distal end 120 also coupled (preferably sewn) to tapered connectors 125 preferably made of hook material. If loop material is used for the outer surface of strap 110, then hook material would be used for the tapered connectors 125. Similarly, if hook material is used for the outer surface of strap 100, then loop material would be used for the tapered connectors 125. The hook and loop material combination is commonly known as VELCRO™, for each material to couple to the other. The tapered connectors 125 are preferably sewn to the distal ends 120 using overlap stitching 130 to reduce any opportunity for the tapered connectors 125 to snag as the tapered connectors 125 of the tracheostomy tie 100 are removed from the tracheostomy tube connectors (not shown) during use. Or, the tapered connectors 125 may be sewn or otherwise attached directly onto and ride the distal ends 120 of strap 110 rather than extend away from them.

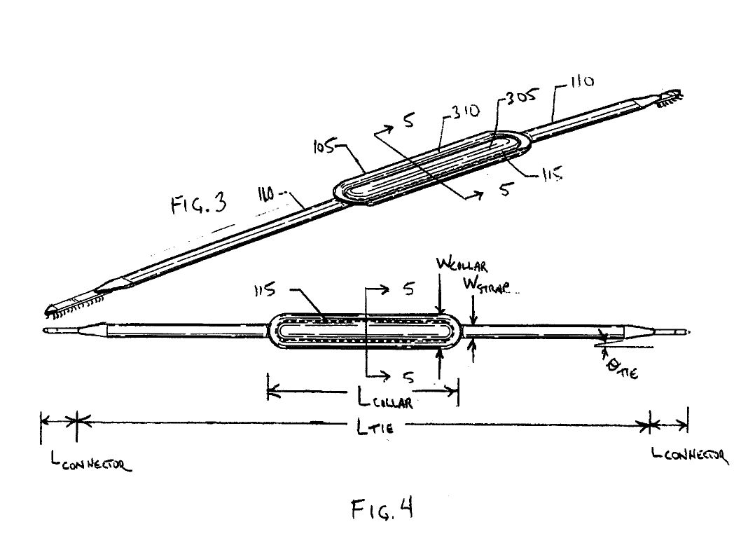

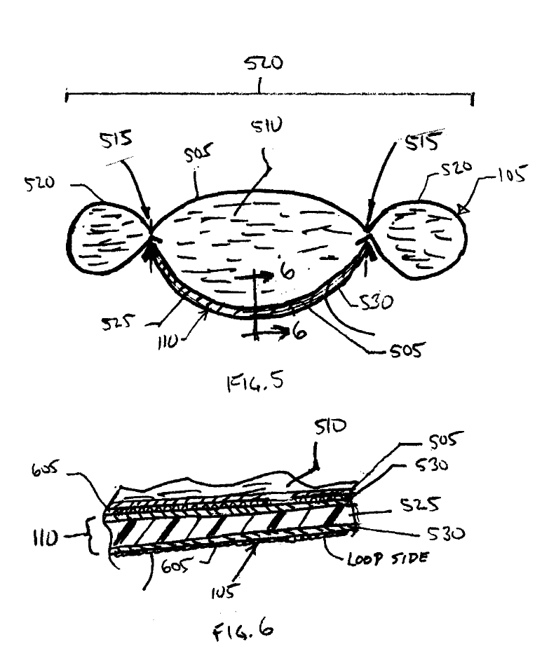

FIGS. 3-4 are perspective views of the tracheostomy tie illustrating a patient neck engagement side, in accordance with one embodiment of the invention. The strap 110 extends from opposite ends of the padded collar 105. The padded collar 105 is preferably sewn to the strap 110 at a location illustrated with dashed sew line 115 to establish a quilted exterior surface for comfortable engagement of the patient's neck. Although such a coupling establishes one longitudinal center pillow 305 and circumferential pillow 310, other quilted patterns may be established with a different sewing pattern. For example, dashed sew line 115 may form other patterns to establish a plurality of pillowed sections. Also, although the padded collar 105 is illustrated as generally rectangular with curved distal ends, the padded collar 105 may have square distal ends or may be itself polygonal or arcuate. In one tracheostomy tie having a θ_{TIE} of approximately 5 degrees and designed for use by a majority of patients, the various elements have the approximate widths and lengths listed in Table 1.

TABLE 1

| | Length |
|---|---|
| | (cm) |
| L_{TIE} | 29-62 |
| L_{COLLAR} | 15-30 |
| L_{CONNECTOR} | 4 |
| W_{COLLAR} | 2 |
| W_{STRAP} | 1 |

In an alternate embodiment, the padded collar 105 may be defined by more than one separate collar (not illustrated) to securely and comfortably attach a tracheal tube to a patient's neck.

FIG. 5 illustrates a cross sectional view of the tracheostomy tie of FIGS. 3-4 as seen along the line 5-5. A collar cover 505, preferably a soft cotton material such as, for example, a 140-200 g type 100% cotton material, encompasses a collar wadding material 510, preferably padded foam having a thickness of approximately 1 cm, to establish the padded collar 105. In the embodiment illustrated in FIG. 5, stitching 515 draws opposite sides of the padded collar 105 together to form a quilted exterior surface 520 that provides a comfortable engagement of the patient's neck without the patient's neck riding on a seam. Also coupled to the padded collar 105 by way of stitching 515 is the strap 110 defined by a strap wadding material, preferably foam material inner layer 525 having a thickness of approximately 3 mm, enclosed by a strap cover 530, preferably double-sided brushed polyester fiber. As the strap 110 experiences stress and resultant strain deformation during use, the collar wadding material 510 enclosed in collar cover 505 serves to resist induced bending moments to reduce changes in the shape of the quilted exterior surface 520. In another embodiment, the padded collar 105 has different materials as between the quilted exterior surface 520 and the side opposite the strap 110.

FIG. 6 is an enlarged cross section view of the padded collar 105 as seen along the line 6-6 of FIG. 5. The foam material inner layer 525 of the strap 110 is enclosed by strap cover 530. The strap cover 530 of the strap 110 is illustrated with loops 605 on its exterior surfaces to illustrate possible complementary operation with hook material on the tapered connector 125 (not shown). Collar wadding material 510 is illustrated adjacent the collar cover 505.

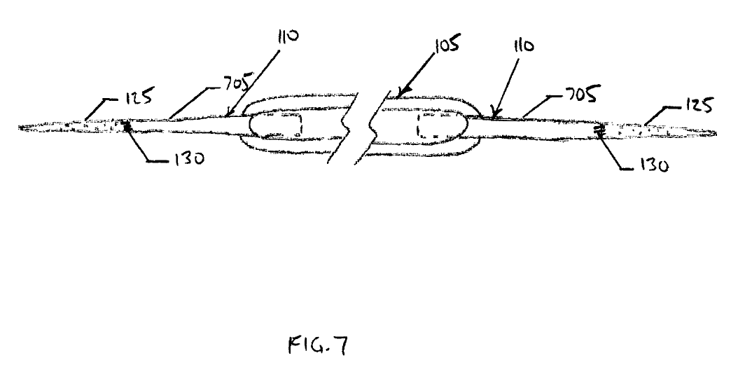

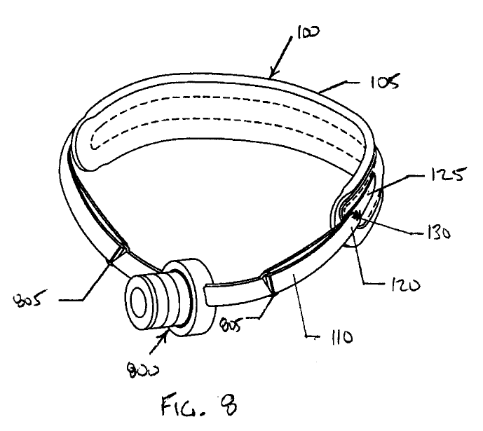

Turning to FIG. 7, an alternative embodiment is illustrated in which the strap 110 is defined by two non-contiguous strap assemblies 705 coupled, preferably sewn, to the padded collar 105 to reduce the quantity of utilized of material. In this embodiment, tapered connectors 125 are also preferably attached to the strap assemblies 705 with the overlap stitch 130 illustrated in FIG. 1 to reduce any opportunity for the tapered connectors 125 to snag as the tapered connectors 125 are removed from the tracheostomy tube connectors (not shown) during use.

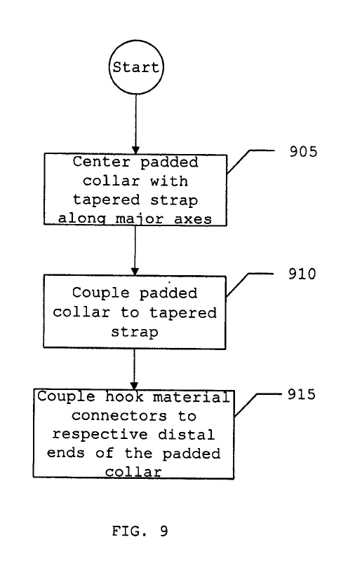

FIG. 8 illustrates a tracheostomy tube assembly 800 previously assembled with the tracheostomy tie of FIGS. 1 and 2. Tapered connectors 125 and distal ends 120 of the strap 110 have been inserted into tracheostomy tube connectors 805 (illustrated as eyelets) and looped back to engage with the loop surface of the padded collar 105. Tapered portions of the tapered connector 125 and distal ends 120 facilitate easy insertion of the strap 110 through the tube connectors 805, while overlap stitching 130 facilitates remove of the same. The strap 110 is preferably sewn to the padded collar 105 at points set back from an outer perimeter of the padded collar 105 to draw the padded collar 105 against a users neck as the tracheostomy tie is positioned and fastened.

FIG. 9 illustrates one embodiment of a method for assembling a tracheostomy tie. Major axes of the padded collar and tapered strap are centered (block 805) and the components are coupled together (block 810), preferably sewn together, to create a quilted section on the padded collar. The quilted section preferably forms center and peripheral pillow surface areas so that a patient's neck rides on the pillows and is not in contact with the seam during normal use. The pillow surface also has the added benefit of allowing better air circulation between the patient's neck and the tracheostomy tie to reduce neck breakdown during normal use. Tapered connectors are coupled to respective distal ends of the padded collar (block 815), preferably with a flush seam. The flush seem creates a smooth transition between the hook material connectors and the padded collar to reduce the possibility of snagging the hook material connectors on a tracheostomy tube connector when a caregiver is removing the hook material connector from the tracheostomy tube connector. The unique combination of a quilted padded collar, tapered strap, tapered connectors and smooth seam stitching between the strap and connectors address both the real-world problem of patient skin breakdown underneath tracheostomy ties during their extended wear and the physical challenge faced by a single caregiver as he or she changes out a tracheostomy tie by themselves.

While various implementations of the application have been described, it will be apparent to those of ordinary skill in the art that many more embodiments and implementations are possible that are within the scope of this invention.

What is claimed, is:

1. A tracheostomy tie apparatus, comprising:
a padded collar having a patient neck engagement side; and
a strap coupled to said padded collar on a side opposite from said patient neck engagement side and extending from opposite ends of said padded collar, said strap comprising:
a loop material outer surface and tapered distal ends; and
tapered connectors connected to respective tapered distal ends of said strap, each of said tapered connectors having a hook material outer surface;
wherein said tapered connectors are operable to pass through respective tracheostomy tube connectors and loop back to engage said loop material so that said padded collar securely fastens and positions a tracheostomy tube against the patient's neck.

2. The apparatus of claim 1, wherein said padded collar comprises:
a collar wadding material; and
a collar cover;
wherein the combination of said collar wadding material and said collar cover provide soft engagement for a patient's neck while resisting bending moments created in said strap in response to stress-strain experienced by said strap during use.

3. The apparatus of claim 2, wherein said collar wadding material comprises a foam material.

4. The apparatus of claim 2, wherein said collar cover comprises a soft cotton material to provide a wicking capability for the patient's neck.

5. The apparatus of claim 2, wherein said collar cover is sewn together adjacent longitudinal sides to enclose said collar wadding material.

6. The apparatus of claim 1, wherein said patient neck engagement side comprises:
a quilted exterior surface to provide comfortable engagement of the patient's neck.

7. The apparatus of claim 1, wherein said strap is coupled to said padded collar on said patient neck engagement side at a plurality of coupling points set back from an outer perimeter of said padded collar.

8. The apparatus of claim 1, wherein said respective tapered connectors are coupled to said respective tapered distal ends with an overlap stitch so that said respective tapered connectors do not snag the tracheostomy tube connector when removed.

9. The apparatus of claim 1, wherein said strap comprises:
a strap wadding material; and
a strap cover to provide a soft engagement with the patient's neck.

10. The apparatus of claim 9, wherein said strap cover comprises a double-sided brushed polyester fiber material to wick moisture from a patient's neck.

11. A tracheostomy tie apparatus, comprising:
a padded collar having an enclosed collar wadding material and forming a quilted patient neck engagement side;
a strap having an enclosed strap wadding material inner layer and a strap cover material selected from the group comprising hook material and loop material, said strap coupled to a back side of said quilted collar and extending from opposite ends of said quilted collar; and
tapered connectors coupled to respective tapered distal ends of said strap, each of said tapered connectors having a connector outer surface material selected from the group comprising hook material and loop material in operative cooperation with said strap cover material, said tapered connectors configured to pass through respective tracheostomy tube connectors and loop back to engage said loop material cover to securely fasten and position a tracheostomy tube about a patient's neck, when a tracheostomy tube is present.

12. The apparatus of claim 11, wherein said strap further comprises two tapered distal ends.

13. The apparatus of claim 11, wherein said strap is coupled to said quilted collar on said patient neck engagement side at a plurality of coupling points set back from an outer perimeter of said padded collar.

14. The apparatus of claim 11, wherein said strap is formed from at least two coupled sections, each of said at least two sections coupled to opposite ends of said padded collar.

15. The apparatus of claim 11, wherein said enclosed collar wadding material is padded foam.

16. The apparatus of claim 11, wherein said enclosed strap wadding material is padded foam.

17. A method of assembling a tracheostomy tie, comprising:
centering the major axis of a padded collar having a first major axis width with the major axis of a tapered strap that has a second width that is less than said first width;
coupling said padded collar to said tapered strap; and
coupling hook material connectors to respective distal ends of said padded collar.

18. The method of claim 17, wherein coupling said padded collar to said tapered strap comprises sewing said padded collar to said tapered strap to create a quilted section.

19. The method of claim 17, wherein said coupling hook material connectors further comprises sewing said hook material connectors to respective distal ends with a flush seam to create a smooth transition between said hook material connectors and said padded collar to reduce the possibility of snagging said hook material connectors on a tracheostomy tube connector when removing said hook material connector from said tracheostomy tube connector.
